(19) United States
(12) Patent Application Publication
(10) Pub. No.: US 2020/0376216 A1
(43) Pub. Date: Dec. 3, 2020

(54) AIRWAY CLEARANCE SYSTEM

(71) Applicants: WILLIAM BEAUMONT HOSPITAL, Royal Oak, MI (US); GRAND VALLEY STATE UNIVERSITY, Allendale, MI (US)

(72) Inventors: Bassel A. SALMAN, Oakland Twp., MI (US); Sam OOSTENDORP, Grand Rapids, MI (US); Jacob D. STEPHENS, Lowell, MI (US); Jordan R. VANDERHAM, Holland, MI (US); Austin J. WILLIAMS, Ada, MI (US); John P. FARRIS, Grand Rapids, MI (US)

(73) Assignees: William Beaumont Hospital, Royal Oak, MI (US); Grand Valley State University, Allendale, MI (US)

(21) Appl. No.: 16/640,228

(22) PCT Filed: Aug. 29, 2018
(86) PCT No.: PCT/US2018/048448

§ 371 Date: Feb. 19, 2020

Related U.S. Application Data
(60) Provisional application No. 62/552,758, filed on Aug. 31, 2017.

(51) Int. Cl. A61M 16/00; A61M 16/20; A61M 16/06

(52) U.S. Cl. CPC A61M 16/0084; A61M 16/209; A61M 2016/003; A61M 2016/0027; A61M 16/06

(57) ABSTRACT

An airway clearance system includes a bladder, a negative pressure relief valve, a positive pressure relief valve, and a port. The bladder is moveable between an expanded state and a compressed state, and defines a first volume in the expanded state and a second volume in the compressed state. The second volume is less than the first volume. The negative pressure relief valve is in fluid communication with the bladder and configured to supply fluid to the bladder from an atmosphere surrounding the bladder. The positive pressure relief valve is in fluid communication with the bladder and configured to supply fluid to the atmosphere from the bladder. The port is in fluid communication with the bladder and configured to supply fluid from the bladder when the bladder moves to the compressed state and to supply fluid to the bladder when the bladder moves to the expanded state.

### CROSS REFERENCE TO RELATED APPLICATION

This patent application claims priority to U.S. Provisional Application Ser. No. 62/552,758 filed Aug. 31, 2017. The entire content of U.S. Provisional Application Ser. No. 62/552,758 is incorporated herein by reference.

### FIELD

The present disclosure relates generally to an airway clearance system, and more particularly to a cough assist system for supplying and removing a fluid from a patient.

### BACKGROUND

This section provides background information related to the present disclosure and is not necessarily prior art.

Individuals suffering from neuromuscular diseases and other ailments often require assistance in cleaning out and clearing their lungs and airways. For example, children and adults suffering from spinal motor atrophy (SMA), cerebral palsy (CP), brain damage, cystic fibrosis, stroke, or chronic obstructive pulmonary disease (COPD) may require airway clearance therapy to dislodge and remove mucus from their lungs. Airway clearance therapy may include applying a series of bursts of pressurized or depressurized (e.g., vacuum) air to or from the patient's lungs or airways, or applying a series of vibrations to the patient's chest. For example, an electronic cough assist device may be used to pressurize or depressurize the patient's lungs and airways by supplying fluid (e.g., air) to and from the electronic cough assist device and the patient's lungs, while a pulmonary vest may be used to apply a series of vibrations to the patient's chest. Such therapy may simulate a cough and help to dislodge or remove mucus from the patient's lungs.

While known airway clearance systems have proven acceptable for their intended purpose, a continuous need for improvement in the relevant art remains.

### SUMMARY

This section provides a general summary of the disclosure, and is not a comprehensive disclosure of its full scope or all of its features.

One aspect of the disclosure provides an airway clearance system. The airway clearance system may include a bladder, a negative pressure relief valve, a positive pressure relief valve, and a port. The bladder may be moveable between an expanded state and a compressed state, and may define a first volume in the expanded state and a second volume in the compressed state. The second volume may be less than the first volume. The negative pressure relief valve may be in fluid communication with the bladder and configured to supply fluid to the bladder from an atmosphere surrounding the bladder when the bladder moves from the compressed state to the expanded state. The positive pressure relief valve may be in fluid communication with the bladder and configured to supply fluid to the atmosphere from the bladder when the bladder moves from the expanded state to the compressed state. The port may be in fluid communication with the bladder and configured to supply fluid from the bladder when the bladder moves from the expanded state to the compressed state and to supply fluid to the bladder when the bladder moves from the compressed state to the expanded state.

Implementations of the disclosure may include one or more of the following optional features. In some implementations, the system includes a gauge operable to measure a pressure or rate of a flow of the fluid through at least one of the negative pressure relief valve, the positive pressure relief valve, or the port.

In some implementations, the negative pressure relief valve includes an adjustable first check valve, and the positive pressure relief valve includes an adjustable second check valve. The adjustable first check valve may be operable to prevent a flow of fluid from the bladder to the atmosphere, and the adjustable second check valve may be operable to prevent a flow of fluid from the atmosphere to the bladder. The adjustable first check valve may include a housing and an adjustment member threadably coupled to the housing. One of the housing and the adjustment member may include an outlet. The adjustable first check valve may include a valve disk and a biasing member. The biasing member may be operable to bias the valve disk into engagement with the one of the housing and the adjustment member to adjust a rate of fluid communication between the bladder and the atmosphere. The adjustment member may be operable to increase a biasing force produced by the biasing member.

In some implementations, the system includes a hinge system and a biasing member. The hinge system may be operable to control a movement of the bladder between the expanded state and the compressed state. The biasing member may be operable to bias the bladder from the compressed state to the expanded state.

In some implementations, the bladder includes an upper plate, a lower plate, and a shell coupled to the upper plate and the lower plate. The upper plate may be parallel to the lower plate in the expanded state and the compressed state. In some implementations, the system includes a retention system configured to secure the bladder in the compressed state. The retention system may include a strap and a key. The strap may include a first end and a second end. The first end may be coupled to one of the upper plate or the lower plate. The key may be coupled to the second end of the strap and operable to be removably secured to the other of the upper plate or the lower plate in the compressed state. The other of the upper plate or the lower plate may include a recess operable to receive the key.

Another aspect of the disclosure provides an airway clearance system. The airway clearance system may include an upper plate, a lower plate, a shell, a hinge system, and a port. The shell may be coupled to the upper plate and the lower plate to define a bladder. The hinge system operable to control a movement of the upper plate relative to the lower plate between an expanded state and a compressed state, and may include a first arm and a second arm. The first arm may include a first end operable to pivot relative to the upper plate and a second end operable to translate relative to the lower plate. The second arm may be pivotally coupled to the first arm and may include a first end operable to pivot relative to the lower plate and a second end operable to translate relative to the upper plate. The port may be in fluid communication the bladder and may be configured (i) to supply fluid from the bladder when the bladder moves from the expanded state to the compressed state and (ii) to supply fluid to the bladder when the bladder moves from the compressed state to the expanded state.

This aspect may include one or more of the following optional features. In some implementations, the system includes an upper rail coupled to the upper plate and a lower rail coupled to the lower plate. The upper rail may include a first slot and the lower rail may include a second slot. The first end of the first arm may be pivotally coupled to the upper rail. The second end of the first arm may be translatably disposed within the second slot. The first end of the second arm may be pivotally coupled to the lower rail. The second end of the second arm may be translatably disposed within the first slot.

In some implementations, the system includes a biasing member having a first end engaging the upper plate and a second end engaging the lower plate. The biasing member may be operable to bias the upper plate away from the lower plate.

In some implementations, the system includes a negative pressure relief valve and a positive pressure relief valve. The negative pressure relief valve may be coupled to the upper plate and in fluid communication with the bladder. The negative pressure relief valve may be configured to supply fluid to the bladder from an atmosphere surrounding the bladder when the upper plate moves from the compressed state to the expanded state. The positive pressure relief valve may be coupled to the upper plate and in fluid communication with the bladder. The positive pressure relief valve may be configured to supply fluid to the atmosphere from the bladder when the upper plate moves from the expanded state to the compressed state.

In some implementations, the upper plate is parallel to the lower plate in the expanded state and the compressed state.

In some implementations, the system includes a retention system configured to secure the upper plate in the compressed state. The retention system may include a strap and a key. The strap may include a first end and a second end. The first end may be coupled to one of the upper plate or the lower plate. The key may be coupled to the second end of the strap and operable to be removably secured to the other of the upper plate or the lower plate in the compressed state. The other of the upper plate or the lower plate include a recess operable to receive the key.

Further areas of applicability will become apparent from the description provided herein. The description and specific examples in this summary are intended for purposes of illustration only and are not intended to limit the scope of the present disclosure.

### DESCRIPTION OF DRAWINGS

The drawings described herein are for illustrative purposes only of selected configurations and not all possible implementations, and are not intended to limit the scope of the present disclosure.

FIG. 1A is a perspective view of an airway clearance system in an expanded orientation in accordance with the principles of the present disclosure, a shell portion of the airway clearance system is shown in broken line format for clarity.

FIG. 1B is a perspective view of the airway clearance system of FIG. 1A in a compressed orientation in accordance with the principles of the present disclosure.

FIG. 1C is a perspective view of the airway clearance system of FIG. 1A in a locked orientation in accordance with the principles of the present disclosure.

FIG. 2 is an exploded view of the airway clearance system of FIG. 1A.

FIG. 3A is a cross-sectional view of a valve system of the airway clearance system of FIG. 1A taken through the line 3A-3A of FIG. 2.

FIG. 3B is a cross-sectional view of a valve system of the airway clearance system of FIG. 1A taken through the line 3B-3B of FIG. 2.

Like reference symbols in the various drawings indicate like elements.

### DETAILED DESCRIPTION

Example configurations will now be described more fully with reference to the accompanying drawings. Example configurations are provided so that this disclosure will be thorough, and will fully convey the scope of the disclosure to those of ordinary skill in the art. Specific details are set forth such as examples of specific components, devices, and methods, to provide a thorough understanding of configurations of the present disclosure. It will be apparent to those of ordinary skill in the art that specific details need not be employed, that example configurations may be embodied in many different forms, and that the specific details and the example configurations should not be construed to limit the scope of the disclosure.

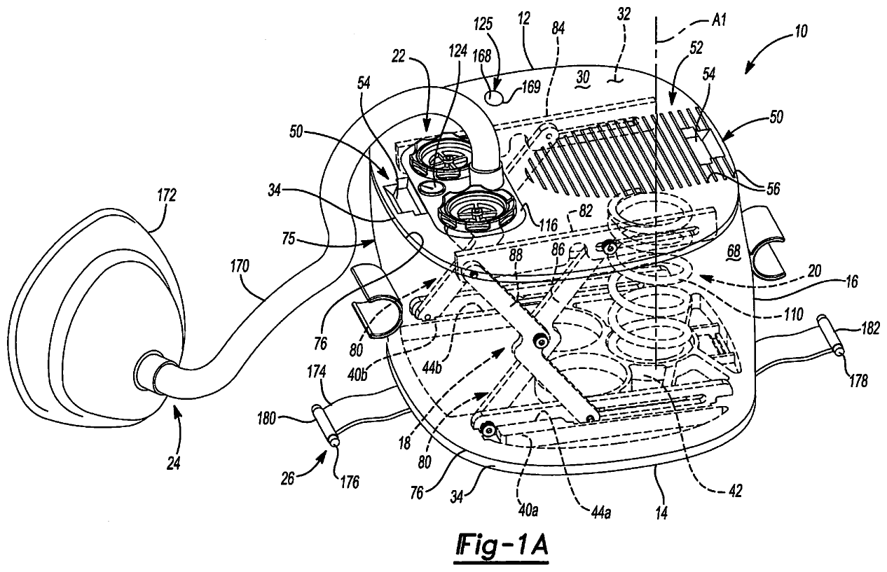

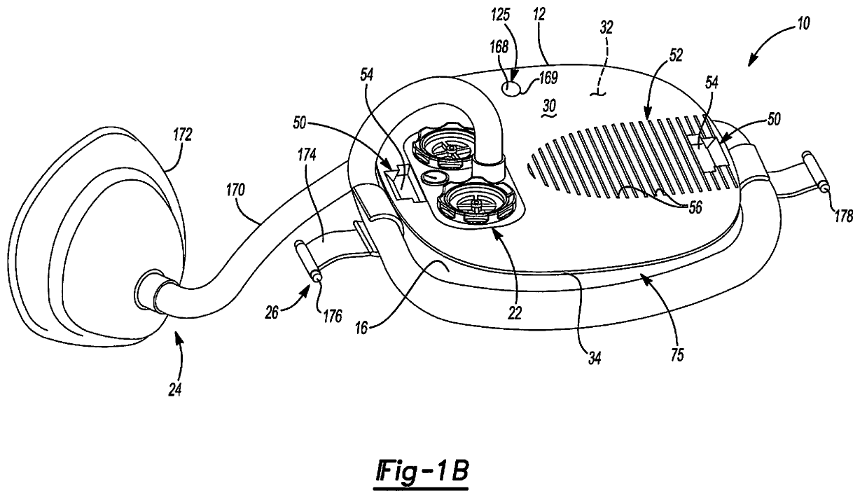

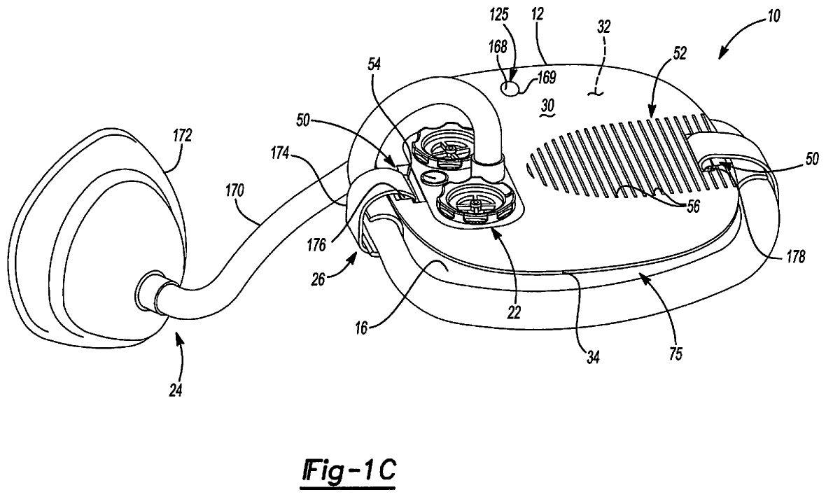

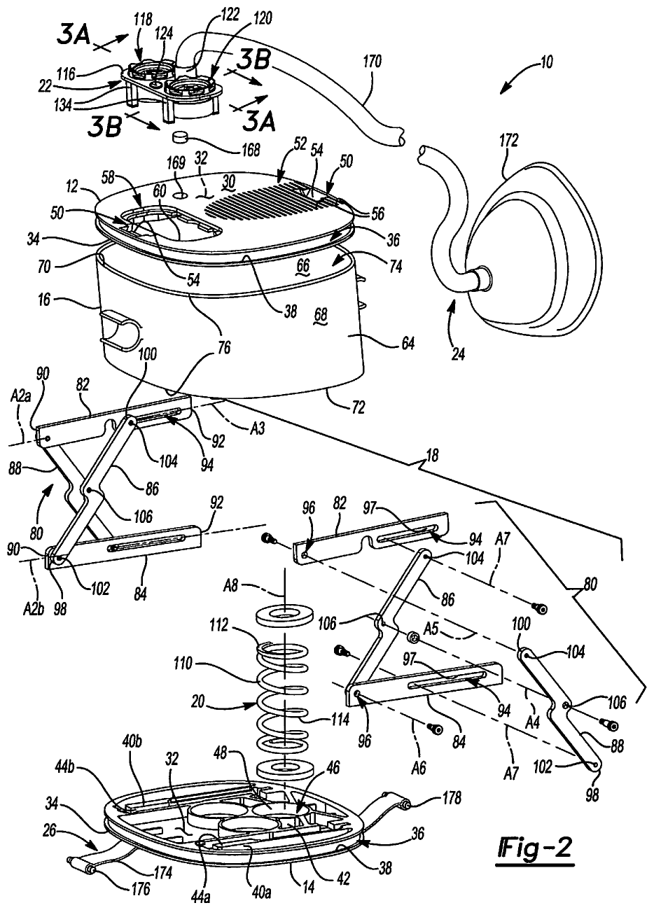

With reference to FIGS. 1A-2, an airway clearance system 10 is illustrated. In some implementations, the system 10 may be used to pressurize or depressurize the lungs and airways of a patient (not shown) by supplying fluid (e.g., air) to and from the system 10 and the lungs and airways of the patient. In particular, the system 10 may deliver fluid (e.g., air) to the lungs of the patient from the system 10 or an atmosphere A surrounding the system, and may deliver fluid to the system 10 or the atmosphere A from the lungs of the patient.

The airway clearance system 10 may include a first or upper baseplate 12, a second or lower baseplate 14, a shell 16, a hinge system 18, a biasing system 20, a valve system 22, a fluid delivery system 24, and a retention system 26.

The upper baseplate 12 may be substantially similar to the lower baseplate 14, except as otherwise shown or described herein. Accordingly, like reference numerals are used hereinafter and in the drawings to identify like features. The upper and lower baseplates 12, 14 may each include an upper surface 30, a lower surface 32 opposite the upper surface 30, and a peripheral wall 34 extending from the upper surface 30 to the lower surface 32. In some implementations, the peripheral wall 34 defines an arcuate (e.g., convex) shape extending about an entirety of the upper or lower baseplate 12, 14. For example, the peripheral wall 34 may define a circular or elliptical shape. As illustrated in FIG. 2, the peripheral wall 34 may be disposed about an entirety of the upper or lower baseplates 12, 14 and may define a shell-receiving feature 36. In some implementations, the shell-receiving feature 36 defines a channel or groove 38 extending around the peripheral wall 34. For example, the groove 38 may be defined in an entirety of the peripheral wall 34.

With reference to FIGS. 1A and 2, the lower surface 32 may include first and second hinge-receiving features 40a, 40b and one or more biasing system receivers 42. The first hinge-receiving feature 40a may be substantially parallel to the second hinge-receiving feature 40b. In some implementations, the first or second hinge-receiving features 40a, 40b define first or second channels 44a, 44b, respectively. The biasing system receiver 42 may define a recess 46 in the lower surface 32. In some implementations, the recess 46 is defined in part by an annular wall 48 (FIG. 2) of the baseplate 12, 14. The annular wall 48 may be concentrically disposed about an axis A1 (FIG. 1A) that intersects the upper and lower baseplates 12, 14 in an assembled configuration.

The upper surface 30 may include one or more retainer-receiving features 50 and an activation feature 52. For example, the upper surface 30 may include two retainer-receiving features 50, each disposed at opposite ends of the upper surface 30. In some implementations, the retainer-receiving features 50 define a cavity 54 in the upper surface 30. The activation feature 52 may include a plurality of ribs 56 extending from the upper surface 30. In some implementations, each rib 56 is substantially parallel to one or more other ribs 56. As will be explained in more detail below, during operation of the system 10, a user may engage the activation feature 52 to operate the system 10.

With reference to FIG. 2, the upper baseplate 12 may also include a valve-receiving feature 58. In some implementations, the valve-receiving feature 58 defines a hole 60 through the upper baseplate 12. In some implementations, the hole 60 defines a stadium shape. It will be appreciated, however, that the hole 60 may define other shapes within the scope of the present disclosure.

The shell 16 may be defined by a body 64 having an inner surface 66, an outer surface 68 opposite the inner surface, a proximal end 70, and a distal end 72 opposite the proximal end 70. The inner surface 66 defines a passage 74 extending through the body 64 from the proximal end 70 to the distal end 72, such that the proximal end 70 forms a proximal opening, and the distal end 72 forms a distal opening. The shell 16 may be formed from an air-tight flexible material (e.g., canvas, fabric, rubber, polymer, or the like).

As illustrated in FIG. 1A, in an assembled configuration, the shell 16 may be coupled to the upper baseplate 12 and the lower baseplate 14 such that the shell 16 and the upper and lower baseplates 12, 14 collectively define a bladder 75. For example, the proximal end 70 of the body 64 may be coupled to the shell-receiving feature 36 of the upper baseplate 12, and the distal end 72 of the body 64 may be coupled to the shell-receiving feature 36 of the lower baseplate 14. In this regard, the system 10 may include a pair of shell retainers 76 to secure the proximal and distal ends 70, 72 of the body 64 within the groove 38 of the upper and lower baseplates 12, 14, respectively. For example, the retainer 76 may include a string, an elastic cord, a hose clamp, or the like, for forming an airtight seal between the body 64 and the peripheral wall 34 of the upper and lower baseplates 12, 14. In some implementations, the retainer 76 is integrally formed with the body 64. In the assembled configuration, the upper surface 30 of the upper baseplate 12 and the upper surface 30 of the lower baseplate 14 may define an angle therebetween. For example, the upper surface 30 of the upper baseplate 12 and the upper surface 30 of the lower baseplate 14 may define an angle between zero degrees and twenty-five degrees. In some implementations, the upper surface 30 of the upper baseplate 12 and the upper surface 30 of the lower baseplate 14 define an angle substantially equal to fifteen degrees to allow a user to more easily and efficiently apply a force on one of the upper surface 30 of the upper baseplate 12 or the upper surface 30 of the lower baseplate 14, while the other of the upper surface 30 of the upper baseplate 12 or the upper surface 30 of the lower baseplate 14 is in a generally horizontal position. As will be explained in more detail below, during operation of the system 10, the angle defined by the upper surface 30 of the upper baseplate 12 and the upper surface 30 of the lower baseplate 14 may remain constant.

The hinge system 18 may include one or more hinge subassemblies 80. For example, as illustrated in FIGS. 1A and 2, in some implementations the hinge system 18 includes two hinge subassemblies 80. It will be appreciate, however, that the hinge system 18 may include more or less than two hinge subassemblies 80 within the scope of the present disclosure.

Each hinge subassembly 80 may include an upper rail 82, a lower rail 84, a first pivot arm 86, and a second pivot arm 88. The upper rail 82 may be substantially similar to the lower rail 84, and the first pivot arm 86 may be substantially similar to the second pivot arm 88, except as otherwise shown or described herein. Accordingly, like reference numerals are used hereinafter and in the drawings to identify like features of the upper and lower rails 82, 84 and of the first and second pivot arms 86, 88. As will be explained in more detail below, during operation of the system 10, the hinge system may control a movement of the upper baseplate 12 relative to the lower baseplate 14 between an expanded state (e.g., FIG. 1A) of the system 10 and a compressed state (e.g., FIG. 1B) of the system 10.

With reference to FIG. 2, the upper and lower rails 82, 84 may each extend along longitudinal axes A2a, A2b, respectively, from a proximal end 90 to a distal end 92. The upper and lower rails 82, 84 may each include a translation feature 94 and a rotation feature 96. The translation feature 94 may extend between the proximal and distal ends 90, 92 along a longitudinal axis A3. In some implementations, the longitudinal axis A3 is substantially parallel to the longitudinal axes A2a, A2b of the respective upper or lower rail 82, 84. As illustrated in FIG. 2, in some configurations, the translation feature 94 defines an aperture or slot 97 through each of the upper and lower rails 82, 84. It will be appreciated, however, that the translation feature 94 may define other constructs (e.g., a rail, a protrusion, or the like) within the scope of the present disclosure. The rotation feature 96 (e.g., an aperture, a hub, or the like) may be disposed at the proximal end 90 of the upper and lower rails 82, 84. In this regard, the translation feature 94 may be disposed between the rotation feature 96 and the distal end 92 of the rails 82, 84.

The first and second pivot arms 86, 88 may each extend from a proximal end 98 to a distal end 100. Each arm 86, 88 may include a first, second, and third rotation features 102, 104, 106. The first rotation feature 102 (e.g., an aperture, a hub, or the like) may be disposed at the proximal end 98. The second rotation feature 104 (e.g., an aperture, a hub, or the like) may be disposed at the distal end 100. The third rotation feature 106 (e.g., an aperture, a hub, or the like) may be disposed between the first and second rotation features 102, 104. For example, the third rotation feature 106 may be centered between the first and second rotation features 102, 104 along the first and second pivot arms 86, 88.

In an assembled configuration, the upper and lower rails 82, 84 may be coupled to the upper and lower baseplates 12, 14, respectively. For example, the upper rails 82 may be coupled to the first and second hinge-receiving features 40a, 40b of the upper baseplate 12, and the lower rails 84 may be coupled to the first and second hinge-receiving features 40a, 40b of the lower baseplate 14. In particular, the upper rails 82 may be disposed within the first and second channels 44a, 44b of the upper baseplate 12, and the lower rails 84 may be disposed within the first and second channels 44a, 44b of the lower baseplate 14, such that the upper and lower baseplates 12, 14 (e.g, the upper surfaces 30) each extend in a direction substantially parallel to the longitudinal axes A2a, A2b of the upper and lower rails 82, 84.

The third rotation feature 106 of the first pivot arm 86 may be pivotally coupled to the third rotation feature 106 of the second pivot arm 88 for rotation about an axis A4, and the first rotation feature 102 of the first and second pivot arms 86, 88 may be pivotally coupled to the rotation feature 96 of the upper and lower rails 82, 84 for rotation about axes A5, A6, respectively. The rotational axes A4, A5, A6 may be substantially parallel to the upper and lower baseplates 12, 14 (e.g, the upper surfaces 30). The second rotation feature 104 of the first and second pivot arms 86, 88 may be translatably or rotatably coupled to the translation feature 94 of the upper and lower rails 82, 84, respectively, for translation along the longitudinal axis A2 of the upper and lower rails 82, 84. For example, a pin of the second rotation feature 104 may be disposed within the slot 97 of the translation feature 94 for rotation about an axis A7 and translation along the longitudinal axes A2a, A2b. The axis A7 may be substantially parallel to the upper and lower baseplates 12, 14 (e.g, the upper surfaces 30) and to the rotational axes A4, A5, A6. Accordingly, as the upper baseplate 12 is moved toward the lower baseplate 14 during operation of the system, the angle defined by the upper surface 30 of the upper baseplate 12 and the upper surface 30 of the lower baseplate 14 may remain constant.

The biasing system 20 may include one or more biasing members 110 extending from a proximal end 112 to a distal end 114 along an axis A8. For example, as illustrated in FIGS. 1A and 2, in some implementations the biasing system 20 includes one biasing member 110. It will be appreciate, however, that the biasing system 20 may include more than one biasing member 110 within the scope of the present disclosure. As illustrated, in some implementations, the biasing member 110 includes a helical compression spring. It will be appreciated, however, that the biasing member 110 may include other forms (e.g., a torsion spring) within the scope of the present disclosure.

In the assembled configuration, the biasing member 110 may be coupled to the upper and lower baseplates 12, 14. For example, the biasing member 110 may be coupled to the biasing system receiver 42 of the upper and lower baseplates 12, 14. In particular, the proximal end 112 of the biasing member 110 may be disposed within the recess 46 of the upper baseplate 12, and the distal end 114 of the biasing member 110 may be disposed within the recess 46 of the lower baseplate 12, such that the annular wall 48 engages, or is otherwise disposed about, the biasing member 110. In this regard, the axis A8 may extend in a direction substantially parallel to the axis A1 and substantially perpendicular to (i) a plane defined by the axes A2a, A2b, A5, A6, or A7 and (ii) the upper and lower baseplates 12, 14 (e.g, the upper surfaces 30).

As illustrated in FIG. 2, the valve system 22 may include a housing 116, an inlet valve 118, an outlet valve 120, a port 122, and a manometer 124. In some implementations, the valve system 22 may also include a release valve 125. As will be explained in more detail below, during operation of the system 10, the valve system 22 may control the flow (e.g., pressure, rate, etc.) of fluid through the port 122 and to the fluid delivery system 24. In the assembled configuration, the valve system 22 may be coupled to one of the upper or lower baseplates 12, 14. For example, the valve system 22 may be coupled to the valve-receiving feature 58 of the upper baseplate 14. In particular, the housing 116 may be sealingly disposed within the hole 60 of the valve-receiving feature 58.

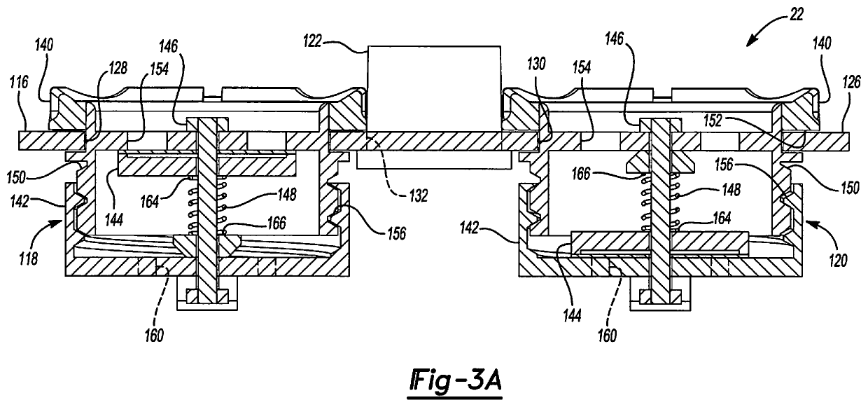

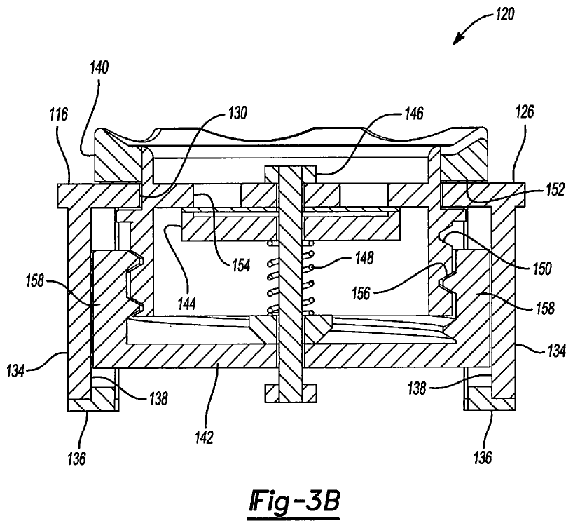

With reference to FIGS. 3A and 3B, the housing 116 may include a body 126 defining first and second apertures 128, 130 and a port 132, and a plurality of legs 134 (FIG. 3B). As illustrated in FIG. 3B, the legs 134 may extend from the body 126 to a distal end 136, and may include a channel 138 extending from and through the distal end 136 and along a length of the leg 134. The port 132 may be in fluid communication with the port 132 and the bladder 75.

The inlet valve 118 may be substantially similar to the outlet valve 120 except as otherwise shown or described herein. Accordingly, like reference numerals will be used to describe like features. As will be explained in more detail below, during operation, the inlet valve 118 may operate to regulate a negative pressure in the bladder 75 and the fluid delivery system 24, and the outlet valve 120 may operate to regulate a positive pressure in the bladder 75 and the fluid delivery system 24. Accordingly, references herein to the negative pressure relief valve and the positive pressure relief valve will be understood to refer to the inlet valve 118 and the outlet valve 120, respectively.

With reference to FIGS. 3A and 3B, the inlet and outlet valves 118, 120 may each include a housing 140, an adjustment member 142, a valve disk 144, a valve needle 146, and a biasing member 148. The housing 140 may include a first thread 150, an annular channel 152, and a plurality of apertures 154 (e.g., inlets or outlets). The adjustment member 142 may include a second thread 156, one or more elongate ribs 158, and a plurality of apertures 160 (e.g., inlets or outlets). In some implementations, the adjustment member 142 includes a pair of elongate ribs 158.

In an assembled configuration, the housing 140 may be rotatably coupled to the housing 116 of the valve system 22, and the adjustment member 142 may be translatably coupled to the housing 116 of the valve system 22. In particular, the housing 116 may be rotatably received within the annular channel 152 and the ribs 158 may be translatably disposed within the channel 138 of the leg 134. The first thread 150 may be threadably coupled to the second thread 156 such that the housing 140 is adjustably (e.g., rotatably) coupled to the adjustment member 142. The valve needle 146 may be coupled to the housing 140 and the adjustment member 142. In particular, the valve needle 146 of the inlet valve 118 may be fixed to the housing 140 and translatably coupled to the adjustment member 142, while the valve needle 146 of the outlet valve 120 may be fixed to the adjustment member 142 and translatably coupled to the housing 140, such that, upon rotation of the housing 140 relative to the adjustment member 142, the housing 140 or adjustment member 142 translates along the valve needle 146. In some implementations, one of the housing 116 or the housing 140 includes one or more protrusions or detents (not shown) and the other of the housing 116 or the housing 140 includes one or more recesses (not shown) to intermittently receive the one or more detents of the housing 116 or the housing 140. In some implementations, the recesses or detents are equally spaced about the apertures 128, 130 or the housing 140. Accordingly, upon rotating the housing 140 relative to the housing 116, the recess(es) may periodically receive the detent(s) to secure the housing 140 relative to the housing 116 in one or more rotational positions, and to indicate an amount (e.g., angle) of rotation of the housing 140 relative to the housing 116.

The valve disk 144 may be coupled to the valve needle 146. For example, the valve needle 146 may be translatably disposed within an aperture of the valve disk 144 and in sealing engagement with the housing 140 (e.g., housing 140 of inlet valve 118) or the adjustment member 142 (e.g., adjustment member 142 of outlet valve 120). In this regard, the inlet and outlet valves 118, 120 may be check valves, such that the valve disk 144 of the inlet valve 118 prevents a flow of fluid from the bladder 75, through the apertures 154 of the housing 140, and to the atmosphere surrounding the system 10, and the valve disk 144 of the outlet valve 120 prevents a flow of fluid from the atmosphere surrounding the system 10, through the apertures 160 of the adjustment member 142, and into the bladder 75.

The biasing member 148 may be supported by the needle 146. For example, in some implementations, the biasing member 148 defines a helical compression spring disposed about the needle 146. In the assembled configuration, a proximal end 164 of the biasing member 148 may engage the valve disk 144, and a distal end 166 of the biasing member 148 may engage one of the housing 140 or the adjustment member 142. Accordingly, upon rotation of the housing 140 relative to the adjustment member 142, the housing 140 or adjustment member 142 translates along the valve needle 146 and adjusts (e.g., increases or decreases) a biasing force produced by the biasing member 148 against the valve disk 144, thereby allowing for an adjustment in the rate of fluid flow through the apertures 154 and into the bladder 75 (e.g., through inlet valve 118) and an adjustment in the rate of fluid flow through the apertures 160 and into the atmosphere surrounding the system 10 (e.g., through outlet valve 120).

The manometer 124, or other suitable gauge operable to measure a characteristic (e.g., pressure, vacuum, flow rate, etc.) of fluid disposed within the chamber or flowing through the ports 122, 132, may be supported by the housing 116, or by the upper or lower baseplate 12, 14, to measure or display a characteristic (e.g., pressure) of the fluid within the bladder 75 and the fluid delivery system 24. For example, as fluid (e.g., air) is forced through the port 132 as the upper baseplate 12 moves along the axis A1 and (i) toward the lower baseplate 14 from an expanded state (e.g., FIG. 1A) to a compressed state (e.g., FIG. 1B), or (ii) away from the lower baseplate 14 from the compressed state (e.g., FIG. 1B) to the expanded state (e.g., FIG. 1A), the manometer 124 may measure and display the pressure within the bladder. In this regard, the user may adjust the inlet or outlet valves 118, 120 (e.g., rotate the housing 140 relative to the adjustment member 142) in order to vary the pressure within the chamber, as measured by the manometer 124, and therefore vary the rate or pressure of the fluid flowing through the port 132. In this way, the system 10 defines a pulmonary flow or loop of fluid through the port 132 to mimic a patient's reverse normal breathing pattern, making the system 10 comfortable for the patient. As used herein, “pressure” may be used to refer to a positive pressure or a negative pressure (e.g., a vacuum).

With reference to FIGS. 1A-2, the release valve 125 may include a plug or cap 168 coupled to one of the upper or lower baseplates 12, 14. For example, as illustrated in FIG. 2, in some implementations, the upper baseplate 14 includes an aperture 169 providing fluid communication between the bladder 75 and the atmosphere surrounding the system 10. In this regard, in the assembled configuration (FIGS. 1A-1C), the cap 168 may be removably disposed within the aperture 169 to prevent fluid communication between the bladder 75 and the atmosphere surrounding the system 10. For example, the cap 168 may be disposed within the aperture 169 in a press- or friction-fit configuration.

As fluid (e.g., air) is forced through the port 132 when the upper baseplate 12 moves along the axis A1 and (i) toward the lower baseplate 14 from an expanded state (e.g., FIG. 1A) to a compressed state (e.g., FIG. 1B), or (ii) away from the lower baseplate 14 from the compressed state (e.g., FIG. 1B) to the expanded state (e.g., FIG. 1A), the positive or negative pressure in the bladder 75 and the fluid delivery system 24 may force the cap 168 of the release valve 125 to be removed from the aperture 169. In particular, when the positive or negative pressure in the bladder 75 exceeds a predetermined threshold, such pressure may overcome the force securing the cap 168 within the aperture 169, thus allowing the bladder 75 to fluidly communicate with the atmosphere through the aperture 169. For example, when the positive pressure in the bladder 75 is greater than thirty centimeters of water, or when the negative pressure in the bladder 75 is less than negative thirty centimeters of water, such pressure may force the cap 168 from the aperture 169, and thus allow the bladder 75 to fluidly communicate with the atmosphere through the aperture 169. In this way, the positive pressure in the bladder 75 can be reduced, or the negative pressure in the bladder 75 can be increased through the aperture 169.

With reference to at least FIG. 2, the fluid delivery system 24 may include a conduit 170 and a mask 172. In the assembled configuration, the conduit 170 may be coupled to the port 122 and the mask 172. Accordingly, during operation of the system 10, the conduit 170 allows for fluid communication between the bladder 75 and the mask 172 through the ports 122, 132.

With reference to FIG. 1A, the retention system 26 may include a strap 174, a first key 176, and a second key 178. The strap 174 may include a flexible member extending from a proximal end 180 to a distal end 182. In some implementations, the strap 174 may be formed from a rigid material defining a C- or U-shape extending from the proximal end 180 to the distal end 182. The first key 176 may include an elongate member coupled to the proximal end 180 of the strap 174, and the second key 178 may include an elongate member coupled to the distal end 182 of the strap 174.

During operation of the system 10, the retention system 26 may be utilized to secure the bladder 75 in the compressed state. For example, as illustrated in FIG. 1C, when the upper baseplate 12 is moved toward the lower baseplate 14, the first and second keys 176, 178 may be coupled to the retainer-receiving features 50 of the upper baseplate 12 to secure the position of the upper baseplate 12 relative to the lower baseplate 14 in the compressed state. In particular, the first and second keys 176, 178 may be disposed within the cavities 54 of the upper baseplate 12.

The foregoing description has been provided for purposes of illustration and description. It is not intended to be exhaustive or to limit the disclosure. Individual elements or features of a particular configuration are generally not limited to that particular configuration, but, where applicable, are interchangeable and can be used in a selected configuration, even if not specifically shown or described. The same may also be varied in many ways. Such variations are not to be regarded as a departure from the disclosure, and all such modifications are intended to be included within the scope of the disclosure.

The terminology used herein is for the purpose of describing particular exemplary configurations only and is not intended to be limiting. As used herein, the singular articles “a,” “an,” and “the” may be intended to include the plural forms as well, unless the context clearly indicates otherwise. The terms “comprises,” “comprising,” “including,” and “having,” are inclusive and therefore specify the presence of features, steps, operations, elements, and/or components, but do not preclude the presence or addition of one or more other features, steps, operations, elements, components, and/or groups thereof. The method steps, processes, and operations described herein are not to be construed as necessarily requiring their performance in the particular order discussed or illustrated, unless specifically identified as an order of performance. Additional or alternative steps may be employed.

When an element or layer is referred to as being “on,” “engaged to,” “connected to,” “attached to,” or “coupled to” another element or layer, it may be directly on, engaged, connected, attached, or coupled to the other element or layer, or intervening elements or layers may be present. In contrast, when an element is referred to as being “directly on,” “directly engaged to,” “directly connected to,” “directly attached to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

The terms first, second, third, etc. may be used herein to describe various elements, components, regions, layers and/or sections. These elements, components, regions, layers and/or sections should not be limited by these terms. These terms may be only used to distinguish one element, component, region, layer or section from another region, layer or section. Terms such as “first,” “second,” and other numerical terms do not imply a sequence or order unless clearly indicated by the context. Thus, a first element, component, region, layer or section discussed below could be termed a second element, component, region, layer or section without departing from the teachings of the example configurations.

What is claimed is:

1. An airway clearance system comprising:
a bladder moveable between an expanded state and a compressed state, the bladder defining a first volume in the expanded state and a second volume in the compressed state, the second volume being less than the first volume;
a negative pressure relief valve coupled to the bladder and in fluid communication with the bladder, the negative pressure relief valve configured to supply fluid to the bladder from an atmosphere surrounding the bladder when the bladder moves from the compressed state to the expanded state;
a positive pressure relief valve coupled to the bladder and in fluid communication with the bladder, the positive pressure relief valve configured to supply fluid to the atmosphere from the bladder when the bladder moves from the expanded state to the compressed state;
a port in fluid communication with the bladder, the port configured to supply fluid from the bladder when the bladder moves from the expanded state to the compressed state and to supply fluid to the bladder when the bladder moves from the compressed state to the expanded state.

2. The airway clearance system of claim 1, further comprising a gauge operable to measure a pressure or rate of a flow of the fluid through at least one of the negative pressure relief valve, the positive pressure relief valve, or the port.

3. The airway clearance system of claim 1, wherein the negative pressure relief valve includes an adjustable first check valve, and the positive pressure relief valve includes an adjustable second check valve.

4. The airway clearance system of claim 3, wherein the adjustable first check valve is operable to prevent a flow of fluid from the bladder to the atmosphere, and wherein the adjustable second check valve is operable to prevent a flow of fluid from the atmosphere to the bladder.

5. The airway clearance system of claim 4, wherein the adjustable first check valve includes a housing and an adjustment member threadably coupled to the housing.

6. The airway clearance system of claim 5, wherein one of the housing and the adjustment member includes an outlet, and wherein the adjustable first check valve includes a valve disk and a biasing member, the biasing member operable to bias the valve disk into engagement with the one of the housing and the adjustment member to adjust a rate of fluid communication between the bladder and the atmosphere.

7. The airway clearance system of claim 6, wherein the adjustment member is operable to increase a biasing force produced by the biasing member.

8. The airway clearance system of claim 1, further comprising:
a hinge system operable to control a movement of the bladder between the expanded state and the compressed state; and
a biasing member operable to bias the bladder from the compressed state to the expanded state.

9. The airway clearance system of claim 1, wherein the bladder includes an upper plate, a lower plate, and a shell coupled to the upper plate and the lower plate.

10. The airway clearance system of claim 9, wherein the upper plate is parallel to the lower plate in the expanded state and the compressed state.

11. The airway clearance system of claim 9, further comprising a retention system configured to secure the bladder in the compressed state.

12. The airway clearance system of claim 11, wherein the retention system includes:
a strap having a first end and a second end, the first end coupled to one of the upper plate or the lower plate; and
a key coupled to the second end of the strap and operable to be removably secured to the other of the upper plate or the lower plate in the compressed state.

13. The airway clearance system of claim 12, wherein the other of the upper plate or the lower plate include a recess operable to receive the key.

14. An airway clearance system comprising:
an upper plate;
a lower plate;
a shell coupled to the upper plate and the lower plate to define a bladder;
a hinge system operable to control a movement of the upper plate relative to the lower plate between an expanded state and a compressed state, the hinge system including:
a first arm having a first end operable to pivot relative to the upper plate and a second end operable to translate relative to the lower plate; and
a second arm pivotally coupled to the first arm and having a first end operable to pivot relative to the lower plate and a second end operable to translate relative to the upper plate; and
a port in fluid communication the bladder, the port configured to supply fluid from the bladder when the bladder moves from the expanded state to the compressed state and to supply fluid to the bladder when the bladder moves from the compressed state to the expanded state.

15. The airway clearance system of claim 14, further comprising an upper rail coupled to the upper plate and a lower rail coupled to the lower plate, the upper rail having a first slot and the lower rail having a second slot, the first end of the first arm pivotally coupled to the upper rail, the second end of the first arm translatably disposed within the second slot, the first end of the second arm pivotally coupled to the lower rail, and the second end of the second arm translatably disposed within the first slot.

16. The airway clearance system of claim 14, further comprising a biasing member having a first end engaging the upper plate and a second end engaging the lower plate, the biasing member operable to bias the upper plate away from the lower plate.

17. The airway clearance system of claim 14, further comprising:
a negative pressure relief valve coupled to the upper plate and in fluid communication with the bladder, the negative pressure relief valve configured to supply fluid to the bladder from an atmosphere surrounding the bladder when the upper plate moves from the compressed state to the expanded state;
a positive pressure relief valve coupled to the upper plate and in fluid communication with the bladder, the positive pressure relief valve configured to supply fluid to the atmosphere from the bladder when the upper plate moves from the expanded state to the compressed state.

18. The airway clearance system of claim 14, wherein the upper plate is parallel to the lower plate in the expanded state and the compressed state.

19. The airway clearance system of claim 14, further comprising a retention system configured to secure the upper plate in the compressed state.

20. The airway clearance system of claim 19, wherein the retention system includes:
a strap having a first end and a second end, the first end coupled to one of the upper plate or the lower plate; and
a key coupled to the second end of the strap and operable to be removably secured to the other of the upper plate or the lower plate in the compressed state.

21. The airway clearance system of claim 20, wherein the other of the upper plate or the lower plate include a recess operable to receive the key.
